(19) United States
(12) Patent Application Publication
(10) Pub. No.: US 2024/0315637 A1
(43) Pub. Date: Sep. 26, 2024

(54) SYSTEM AND METHOD FOR WHOLE-BODY BALANCE ASSESSMENT

(71) Applicant: Hoffmann-La Roche Inc., Little Falls, NJ (US)

(72) Inventors: Ralf Josef JAEGER, Basel (CH); Xing CHEN, Basel (CH)

(73) Assignee: Hoffmann-La Roche Inc., Little Falls, NJ (US)

(21) Appl. No.: 18/576,270

(22) PCT Filed: Jul. 1, 2022
(86) PCT No.: PCT/EP2022/068330

§ 371 Date: Jan. 3, 2024

(30) Foreign Application Priority Data
Jul. 6, 2021 (EP) 21183926.1

(51) Int. Cl. A61B 5/00; A61B 5/11

(52) U.S. Cl. CPC A61B 5/4023; A61B 5/0013; A61B 5/0077; A61B 5/1128; A61B 5/7203; A61B 5/7246; A61B 5/7278; A61B 2576/00

(57) ABSTRACT

Systems and methods for analyzing whole-body balance comprise and perform the steps of: obtaining depth image data of a person's body during a predefined time period; tracking locations of a plurality of anatomical landmarks of the body based on the depth image data; estimating a mass center of the body based on a tracked location of each of one or more of the anatomical landmarks; and determining a stability of body, SoB, score based on a deviation of the estimated mass center over the predefined time period, wherein the SoB score is indicative of the whole-body balance.

### TECHNICAL FIELD

The present application generally relates to the field of whole-body balance assessment.

### BACKGROUND

Recent progress in image processing allows to track the movement of pre-defined parts of body over time by means of a camera sensor and a computing device.

For instance, Microsoft's Kinnect™ sensor includes an RGB video camera, microphones and an infrared sensor and allows to obtain depth images. It further allows to perform gesture recognition, speech recognition, and body skeletal detection. It is commonly used to mediate an interaction between a human and a computing device, for instance in context of gaming, and for unobtrusive movement analysis of a human body.

The evolution of sensor technology has also enabled the healthcare community to use digital tools to generate real-world data and real-world evidence. Accurate assessments of spatial and temporal characteristics of human balance are important diagnostic and therapeutic information. Such information can help to diagnose diseases accompanied with movement disorders, such as neurodegenerative diseases and neuromuscular diseases. Examples are Huntington's disease, Parkinson's disease, and Primary Lateral Sclerosis.

Clinical rating scales to assess and quantify the progression of neurodegenerative illnesses characterized by disorders of movement, cognition, behavioral abnormalities, and functional capacity exist. For example, the Unified Huntington's Disease Rating Scale, UHDRS, which is a test specifically developed to assess the progression of Huntington's Disease. The test to determine UHDRS has four parts, namely (1) motor function including 31 items with a 5-point ordinal scale ranging from 0-4, (2) cognitive function assessment, (3) behavioral assessment, and (4) functional capacity reported as the Total Functional Capacity Score, TFC. UHDRS characteristics of each part are assessed typically manually by domain experts, e.g., physicians over time for individual patients. The conventional assessment of UHDRS is therefore a complex procedure which is work- and cost intensive. Since the test is done by different domain experts for different patients at different times, the consistency and comparability of test results determined by different domain experts is naturally afflicted with uncertainty or bias, which is undesirable. Details of the of UHDRS test characteristics are described by the Huntington Study Group in “Unified Huntington's Disease Rating Scale: Reliability and Consistency”, in Movement Disorders, Vol. 11, No. 2, 1996, pp. 136-142 available at https://www.movementdisorders.org/MDS-Files1/PDFs/Rating-Scales/uhdrs.pdf.

### SUMMARY

A simplified summary of some embodiments of the disclosure are provided in the following to give a basic understanding of these embodiments and their advantages. Further embodiments and technical details are described in the detailed description presented below.

According to an embodiment, a computer-implemented method for analyzing whole-body balance of a person, the method comprising the steps of obtaining depth image data of a person's body during a predefined time period; tracking locations of a plurality of anatomical landmarks of the body based on the depth image data; estimating a mass center of the body based on a tracked location of each of one or more of the anatomical landmarks; and determining a stability of body, SoB, score based on a deviation of the estimated mass center over the predefined time period, wherein the SoB score is indicative of the whole-body balance. This has the advantage that whole-body balance can be assessed objectively.

In one embodiment, the center of mass is estimated by a sum vector of each of the one or more anatomical landmarks relative to a predefined anatomical landmark in one, two, or three spatial dimensions. This has the advantage that the stability of balance score has strong correlation with presence and severity of movement disorders of the person that can be related to neurodegenerative or neuromuscular diseases.

In one embodiment, each anatomical landmark corresponds to a joint of the person's skeleton and the predefined anatomical landmark corresponds to a spine base, a hip center, or a pelvis center of the body. This provides the advantage that the common software development kit, SDK, which allow tracking of skeleton joints, can be used for the balance assessment.

In one embodiment, the SoB score is a standard deviation of the center of mass estimation over the predefined time period.

In one embodiment, the method further comprises excluding one or more of the plurality of anatomical landmarks based on a noise level of the tracked locations of respective anatomical landmarks and/or smoothing a series of tracked locations of one or more of the plurality of anatomical landmarks. This has the advantage that even inaccuracy and noises in the recording of depth image data can be tolerated, without scarifying accuracy of the final SoB score. Inaccuracy and noise in the depth image data and resulting movement data can e.g. be caused by the quality of the motion sensor or by inadverted movement of the tested person.

in one embodiment, the method further comprises recording a plurality of SoB scores of the person over time in a data store to assess, using Spearman correlation, a progression of neurodegenerative or neuromuscular disease of the person and/or to monitor the effect of one or more treatment schemes such as medication and physiotherapy.

In one embodiment, obtaining depth image data of the person's body includes capturing the depth image data using a motion sensor.

According to a further embodiment. a system for analyzing whole-body balance of a person comprises: a motion sensor for capturing depth image data of a person's body and a processor in communication with the motion sensor, wherein the processor is configured to perform the steps of: tracking locations of a plurality of anatomical landmarks of the body based on the depth image data; estimating (540) a mass center of the body based on a tracked location of each of one or more of the anatomical landmarks; and determining a stability of body, SoB, score based on a deviation of the estimated mass center along the predefined time period, wherein SoB score is indicative of the whole-body balance.

In one embodiment, the system further comprises a data store communicatively connected to the processor, wherein the processor is further configured to perform the step of recording a plurality of SoB scores of a person overtime in the data store to assess, using Spearman correlation, a progression of neurodegenerative disease and/or to monitor the effect of one or more treatment schemes such as medication and physiotherapy.

In one embodiment, the motion sensor is a 3D motion sensor including an infrared camera, a Kinect sensor, or a RealSense sensor. This has the advantage, that the system and setup of a test environment is simple and can be achieved with commodity components in an office setting.

In one embodiment, the motion sensor is included in a mobile computing device. This aspect simplifies the deployment and execution of the whole-body balance assessment and has the advantage that balance assessment described herein can be carried out using a simple personal health care application on the mobile device.

In one embodiment, the processor is included in a remote computer and the depth image data is communicated by the mobile computing device via a network to the remote computer. This has the advantage, that compute-intensive operation of carrying out the whole-body balance assessment can be performed on the remote computer.

In one embodiment, a server computing device comprises a network adapter; and a processor adapted to perform the steps: receiving depth image data of a person's body via the network adapter; tracking locations of a plurality of anatomical landmarks of the body based on the depth image data; estimating a mass center of the body based on a location of each of one or more of the anatomical landmarks; and determining a stability of body, SoB, score based on a deviation of the estimated mass center along the predefined time period, wherein the SoB score is indicative of the whole-body balance of the person's body.

In one embodiment, a computing device is a mobile device and comprises a motion sensor and a processor adapted to perform the steps according to the method for assessing whole-body balance according to one of the methods specified hereinabove.

In a further embodiment, a computer-readable medium comprises instructions which, when executed by a computer, cause the computer to carry out the method for analyzing whole-body balance of a person according to the methods specified hereinabove.

The present technology solves the problem of evaluating, preferably in real-time, the stability of a human body through a balance test without a sophisticated setup. The whole-body movement can be detected, tracked, and analyzed in an easy and straightforward way. The balance tests described herein are relatively simple to set up and can be carried out in an office environment. They required components typically have low-cost.

The foregoing general description and the following detailed description are exemplary and explanatory only and are not restrictive of the invention as claimed.

### BRIEF DESCRIPTION OF THE DRAWINGS

The foregoing summary as well as the following detailed description of preferred embodiments are better understood when read in conjunction with the append drawings. For illustrating the invention, the drawings show exemplary details of systems, methods, and experimental data. The information shown in the drawings are exemplary and explanatory only and are not restrictive of the invention as claimed. In the drawings:

FIG. 1 shows a block diagram of a system for analyzing whole-body balance;

FIG. 2 shows a block diagram of a system for analyzing whole-body balance including a client and a server;

FIG. 3 shows the spatial orientation of a system for analyzing whole-body balance;

FIG. 4 shows a model of person's skeleton including joints;

FIG. 5 shows a flowchart computer-implemented method for analyzing whole-body balance.

FIG. 6 is a plot of statistical data showing mass center deviation in x direction for individuals in different classes including patients with Huntington's Disease (HD), patients with Parkinson's Disease (PD), patients with Primary Lateral Sclerosis (PLS) and healthy persons in a control group (CON);

FIG. 7 is a plot of statistical data showing mass center deviation in y direction for individuals in different classes including patients with Huntington's Disease (HD), patients with Parkinson's Disease (PD), patients with Primary Lateral Sclerosis (PLS) and healthy persons in a control group (CON);

FIG. 8 is a plot of statistical data showing mass center deviation in z-direction for individuals in different classes including patients with Huntington's Disease (HD), patients with Parkinson's Disease (PD), patients with Primary Lateral Sclerosis (PLS) and healthy persons in a control group (CON);

FIGS. 9a to 9h are plots showing the Spearman correlation and significance between mass center movement in z and x direction and UHDRS components including total motor scores (motscore), TFC, trunk chorea (chortrnk) and retropulsion pull test (retropls);

FIGS. 9i to 9p are plots showing the Spearman correlation and significance between features of whole-body movement in z and x direction and UHDRS components including total motor scores (motscore), TFC, trunk chorea (chortrnk), and retropulsion pull test (retropls).

### DETAILED DESCRIPTION

The present disclosure relates to methods and systems for a computer-implemented analysis for assessing whole-body balance. The assessment can, different from an assessment done solely by a human, be objective and reproducible. This is an important property and prerequisite for accurate diagnosis and monitoring in movement disorders. Movement disorders can occur for example in the context of neurodegenerative diseases or neuromuscular diseases. Neurodegenerative diseases are for example Huntington's disease (HD), Parkinson's disease (PD) or Primary Lateral Sclerosis (PLS).

The methods and systems disclosed herein permit do determine the severity of a movement disorder and track the progression of the severity over time. In the context of a therapy, the effects of a treatment method on a patient, which may be medication, physiotherapy or other forms of therapy can be estimated and tracked over time.

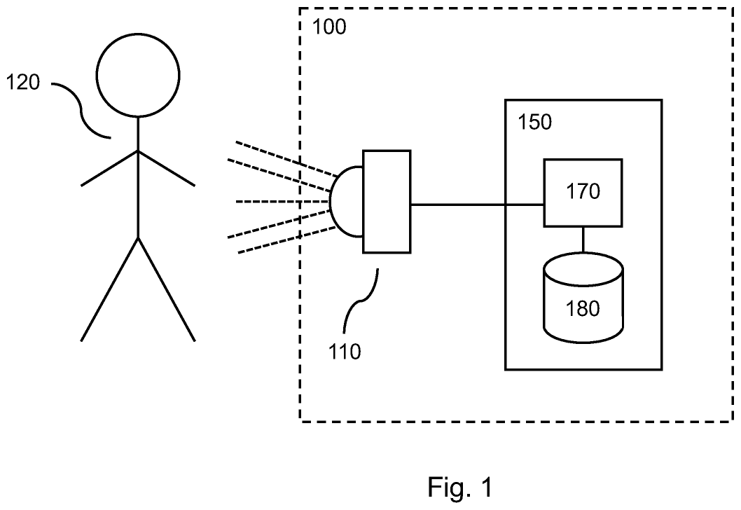

FIG. 1 shows a system 100 for analyzing whole-body balance according to an embodiment. A person's body 120 is located in the field of view of a motion sensor 110.

The motion sensor 110 can be a three-dimensional motion sensor and may include, for example, an infrared depth camera. Examples of commercial sensors are Microsoft's Kinect™ sensor and Intel RealSense sensor. These and other sensors are marker-fee 3D motion sensors, which means that they do, not require that the person, for which movement is to be determined, is not required to wear body markers, which simplifies the test setup.

The motion sensor 110 is connected to computing device 150 that includes a processor 170, configured to run software, which is typically based on a software development kit, SDK, provided with the motion sensor 110 to obtain and process data from the motion sensor 110. In that manner, the processor 170 is capable to obtain from data corresponding to individual depth images information about the location of anatomic landmarks of the person's body 120. The real time locations of anatomical landmarks can for instance be obtained with the Microsoft Software Development Kit or other software packages based on commercially available depth cameras such as Microsoft Kinect™.

The motion sensor 110 and the computing device 150 may be integrated in a single device or they may be separate devices that are communicatively coupled.

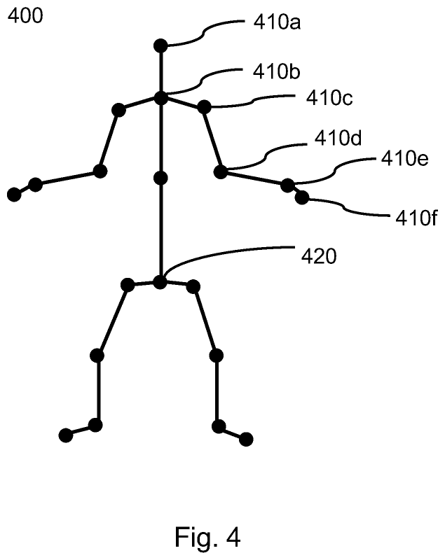

In some examples anatomic landmarks can be skeleton joints, also called joints or join points of the person's body, or body points. The kind of landmarks available is typically defined by the SDK provided with the motion sensor 110. For example, the motion sensor 110 in combination with an SDK can provide three-dimensional locations of skeleton joints in real-time. Skeleton joints are discussed in the context of FIG. 4.

FIG. 4 shows a person's skeleton 400. Anatomical landmarks provided by common motion sensor SDKs include two ankles, two knees, two hips, two shoulders, two elbows, two wrists, spine middle, neck, and head. Some of the skeleton joints are indicated in the FIG. 4 as black dots. 410a denotes the head, 410b denotes the neck, 410c denotes a shoulder, 410d denotes an elbow, and 410e denotes a hand wrist, 410f a hand.

Predetermined landmark 420 denotes the spine base or a hip center 420. Typical frameworks provide at least 20 anatomical landmarks illustrated in FIG. 4.

Depth image data is obtained 510 through the motion sensor from the person's body 120 during a predefined time period. For a typical whole-body balance assessment, a predefined time period of 30 seconds is commonly used. For example, the motion sensor 110 may capture depth image data during this time period at a rate a rate of 30 Hz and provide the same to the processor for analysis.

The analysis of depth data captured during the predefined time period provides motion information for each individual anatomic landmark, and more specifically each skeleton joint, during the predefined time period. For example, using the SDK, information is provided, for each anatomic landmark, about the spatial position of the landmark at each time of a frame capture during the predefined time period.

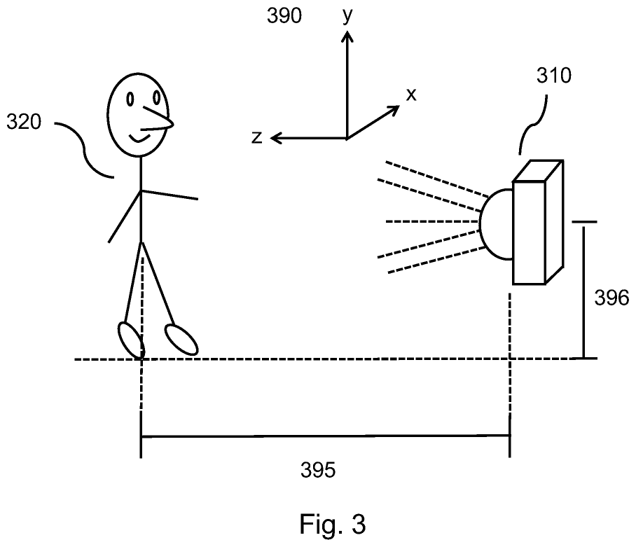

FIG. 3 illustrates details about the spatial information and orientation of the person's body 320 in relation to the motion sensor 310. Spatial coordinates are described by the coordinate system 390, in which the x-axis denotes the horizontal direction, the y-axis denotes the vertical direction, and the z-axis denotes the direction between the position of a person's body 320 and the position of a 3D-motion sensor 310. Spatial information includes information about coordinates in x, y, z direction.

The person's body 320 may be located at a predetermined distance dist to the motion sensor. This distance 395 between the person 320 and the motion sensor 310 can depend on the type and characteristics of the motion sensor 310 and is typically between 0.8 m to 4 m, as long as the whole body is in the field of view of the motion sensor 310. For a typical balance test, a person is instructed to stand still during the predefined time period of 30 seconds at a distance such that whole body is in the field of view of the motion sensor 310, which is commonly of about 3.5 meters away from the motion sensor 310.

The motion sensor 310 is preferably positioned at a predetermined height h. This height 396 above the ground is, for example, around 0.8 m, which is the typical height when the motion sensor is positioned on a table.

The information, for each anatomic landmark, about the spatial position of the landmark at each time of a frame capture during the predefined time period is used for assessment of the person's whole-body balance as described herein in the following. The assessment procedure based on motion data captured during a single predefined time period for an individual person is referred to as an individual whole-body balance assessment.

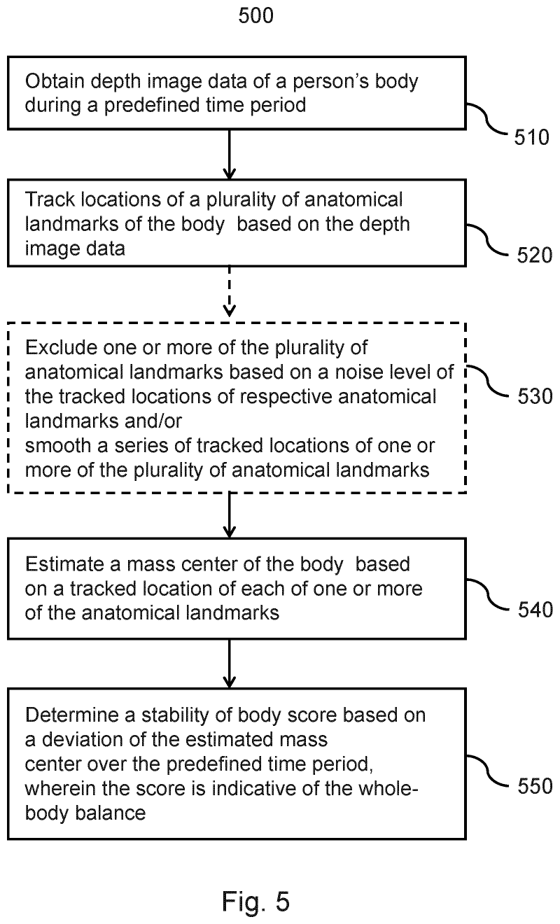

The processor 170 of computing device 150 is configured to perform the steps for analyzing whole-body balance according to the method described in the context of FIG. 5 hereinafter.

The computing device 150 may further include a data store 180 to record of motion data obtained over a longer period of time during a plurality of individual whole-body balance assessments of the same or different persons. Analysis of historical movement data and information derived through further analysis thereof, can, for example, be used to assess progression of neurodegenerative disease and/or to monitor the effect of a treatment method on an individual person, which may be medication, physiotherapy, or other forms of therapy, over time.

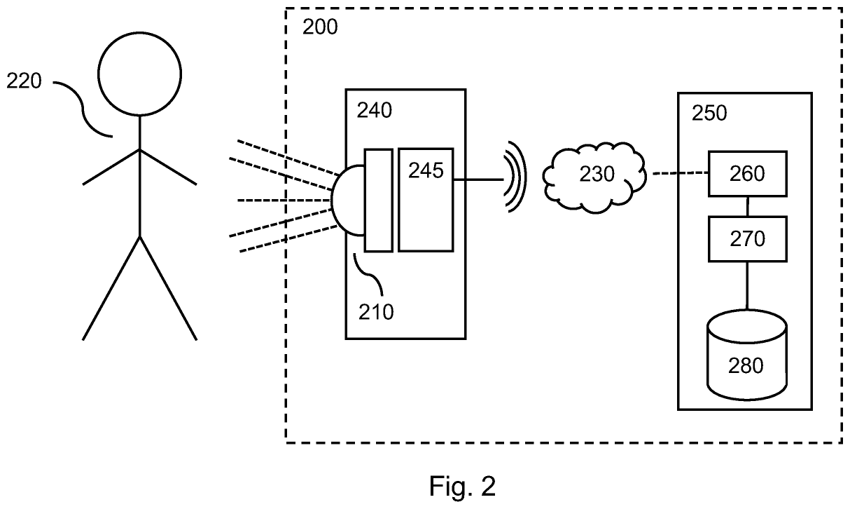

FIG. 2 shows a system 200 for analyzing whole-body balance according to another embodiment. The system 200 differs from the system 100 only in that it is organized as a client-server architecture. For example, the location of the person 220 in relation to the motion sensor 210 corresponds to the arrangement discussed above for the system 100.

The system 200 comprises a server computing device 250 in communication with a network 230. The server device includes a network interface 260 communicatively coupled to the network 230. Server computing device may be implemented as virtual machine and/or can be hosted in a cloud computing environment. The processor 270 is adapted to receiving depth image data of a person's body 220 via the network adapter 260 through the network 230. Depth image data is received through the network 230 from a client computing device 240.

Said client computing device 240 includes a processor 245. A motion sensor 210 as described above with reference FIG. 1 can be connected or can be included in the client computing device 240. The processor 245 is configured to control and run the motion sensor 240 and received depth data therefrom.

Client computing device 240 is connected to a network 230 by a wireless connection. Alternatively, not shown in FIG. 2, client computing device 240 can be connected to the network 230 by a wire connection. Client computing device 240 can be a mobile computing device, such as smartphone or handheld tablet computer. The client computing device can a user device be configured with a personal digital health application, which is a computer program including one or more program instructions, that when executed by the processor 245 of the client computing device 240, cause the device 240 to perform whole-body balance assessment according to the steps illustrated in the context of FIG. 5 and described hereinafter.

The client computing device 240 can be configured to transfer the depth image data captured by the motion sensor 210 during a predetermined period of time as described above for the embodiment according to system 100. In this case, the client device 240 is configured to transfer the depth image data for an individual whole-body balance assessment to the server computing device 250, the processor 270 of which performs further subsequent analysis of the depth image data of said individual assessment related to whole-body balance assessment according to the steps illustrated in FIG. 5 and described hereinafter. In this case, the server computing device 250 is an embodiment for analyzing whole-body balance according to the present disclosure.

Alternatively, the client computing device 240, specifically its processor 245, can be configured to perform further subsequent analysis steps related to whole-body balance assessment according to the steps illustrated in FIG. 5 and described hereinafter. In this case, the client computing device 240 is an embodiment for analyzing whole-body balance according to the present disclosure.

The computing device 250 may further include a data store 280 to record motion data obtained over a longer period of time during a plurality of whole-body balance assessments of different persons. Analysis of historical movement data and information derived through further analysis thereof, can, for example, be used to assess progression of neurodegenerative disease and/or to monitor the effect of a treatment method on an individual person, which may be medication, physiotherapy, or other forms of therapy, over time.

In the system 200 based on a client-server architecture, the server computing device 250 may communicate results of an individual whole-body balance assessment and/or results from the analysis of historical whole-body balance assessments stored on the data store 280 of an individual person back to the client computing device 240.

Processors 170 of system 100 and processors 245 or 270 of the system 200 can be configured to carry out operations to perform steps for analyzing whole-body balance illustrated in FIG. 5 and as described hereinafter.

The technology according to the present disclosure can also be embodied in a computer-readable medium comprising instructions which, when executed by a computer, cause the computer to carry out operations to perform steps for analyzing whole-body balance illustrated in FIG. 5 and as described hereinafter.

FIG. 5 is a flow chart of a computer-implemented method 500 for analyzing whole-body balance according to an embodiment of the present disclosure. The method starts with the step of obtaining 510 depth image data of a person's body during a predefined time period. The depth data is acquired by a motion sensor as described hereinabove and may be transferred through a network in client-server environment as explained hereinabove. For a typical whole-body balance assessment, a predefined time period of 30 seconds is appropriate. For example, the motion sensor may capture depth image data during this time period at a rate a rate of 30 Hz, resulting in a respective number of frames per second, forming time steps, and provide the same to the processor for analysis. In this analysis, which typically performed by an SDK provided for the motion sensor, e.g. by developers of the motion sensor, by third parties, or open source, anatomical landmarks of a person's body in the field of view of the motion sensor are recognized in each depth image during the predetermined period of time. The detection is performed using advanced image processing and detection techniques. As a result, for each time step t during the predefined time period, information about a plurality of anatomical landmarks is provided, the information of each anatomical landmark comprising coordinates 330 in three spatial dimensions x, y, z (also referred to a directions) that specify the location of the person's body point corresponding to the anatomical landmark at time t.

In a subsequent step of method 500, locations of a plurality of anatomical landmarks of the body based on the depth image data, more specifically on the location information about a plurality of anatomical landmarks provided at each time step t, are tracked 520 over all time steps during the predefined time period. In that manner, movement of individual body parts corresponding to the anatomical landmarks can be determined.

Anatomical landmarks 410, 420 used herein and shown in FIG. 4, are for example, skeleton joints including two ankles, two knees, two hips, two shoulders, two elbows, two wrists, spine middle, neck, and head. Comprehensive information revealing the spatiotemporal characteristics such as variance can be derived from the real-time locations of these body points.

The movement data tracked in step 520 is intended for use in a balance test as described in the following. Tracking results, for example, in a series of spatial locations for each anatomical landmark. Such data is represented, for example, as a vector specifying movement data for an individual anatomical landmark as a series of locations, the vector comprising data records, each including information about the time at which the data was captured and spatial coordinates x, y, z of the location of the anatomical landmark.

The method includes, optionally a quality control step to ensure the correct balance capturing. The quality control step includes one or more of the following quality control checks.

One check relates to the body height. An estimated body height is determined based on the tracked skeleton joints, which is compared to an indicated real body height of the person undergoing balance assessment. If there is substantial correspondence between both height measures, it is likely that a substantial portion of the anatomical skeleton is taken for collecting the movements. In that manner, it can also be ensured, that no other moving or static items in the field of view of the motion sensor are captured and falsely identified as skeleton joints.

Another check is directed to assure that the whole tracked skeleton is within the field of view of the motion sensor and remains so during a particular balance assessment. If it is determined that only a partial capture occurred, e.g. that only a subset of the skeleton joints is obtained by the tracking based on the depth image data, then movements determined from such data should not be taken for balance analysis.

A third check relates to ensure that a “clean” balance test is captured instead of being interfered by other movement such as walking by comparing the difference of the standing and ending position. For example, if the movement data is determined to show patterns indicating, e.g., clear walking movements, the data may be rejected for testing and a new set of depth image data may be obtained according to step 510 above.

Alternatively, identified walking steps and corresponding depth image data or movement data obtained from tracked locations of anatomical landmarks during walking can be removed such that only “clean” data is used for subsequent steps of estimating mass center and further analysis.

More generally, if any of the one of more of the above checks fail or do not meet a predefined threshold criterium, the method may restart overall and obtain a new set of depth image data according to step 510 etc.

Optionally, certain anatomical landmarks may be excluded 530 from the tracking based on a noise level of specific body points in the individual dataset, i.e. the depth image data captured during a certain balance assessment of a person. For example, body points, that have higher noise levels than other body points, may be excluded. The exclusion can be predetermined, such that certain landmarks are generally never used for subsequent steps in the show-body balance assessment analysis or based on noise levels determined for tracked anatomical landmarks in an individual data set. For example, skeleton joints related to hands 410f and feet may be excluded, since these body points may commonly indicate higher degrees of movement relative to other body points and corresponding anatomical landmarks that indicate less movement. For example, since wrists and ankles are used, hands and feet provide basically redundant information of distal movement which is not highly related to the whole-body balance. In addition, distal locations have higher uncertainty (noise). Moreover, pronounced movement of hands and feet can be unrelated to whole-body balance and therefor inclusion of movement data of these anatomical landmarks in the assessment of whole-body balance may distort overall analysis results. Exclusion is limited in the sense that a certain minimum number of body points must be available to ensure that subsequent analysis steps are meaningful and that a reliable SoB score results.

The method 500 continues with estimating 540 a mass center of the body based on a tracked location of each of one or more of the anatomical landmarks as far as they have not been excluded in step 530. For example, the center of mass can be estimated 540 by a sum vector of each of the one or more anatomical landmarks relative to a predefined anatomical landmark in one, two, or three spatial dimensions. The predefined anatomical landmark can be understood as a reference landmark. Clinical tests demonstrated that a predefined anatomical landmark corresponding to, for example, the spine base, hip center, or pelvis center of the body (320), result in most accurate estimation of the mass center of the body and most accurate balance assessment.

The method 500 continues by determining 550 a stability of body, SoB, score based on a deviation of the estimated mass center over the predefined time period, wherein the SoB score is indicative of the whole-body balance. The SoB score may be a standard deviation of the center of mass estimation over the predefined time period.

The SoB score is calculated according to the formula:

$$S o B = {σ}_{t} ( {Σ}_{j} ( {P}_{j , d , t} - {P}_{predef , d , t} ) ) .$$

P_{j,d,t} denotes the joint position, outputted by the motion sensor and the software development kit, and determined by the tracking step, j specifies to which joint of the plurality of joints it is referred to. d specifies the dimension, whereby x may refer to the horizontal direction, y may refer to the vertical direction, and z may refer to the direction of the camera, as shown in FIG. 3. A person skilled in art realizes without any doubt that the individual directions can be denoted by different signs, and coordinate systems that are different from cartesian coordinates may be used. t indicates the specific time during the predefined time period at which the location data of joint j is determined.
P_{predef,d,t} denotes the position of the predefined anatomical landmark in dimension d and at time t. In this description, P_{predef,d,t} refers to the spin base unless stated differently.
Σⱼ denotes the sum vector of each joint relative to the spine base in each direction and in a specific time frame.
σₜ denotes the standard deviation over all data points at times t over the predefined time period during which the balance assessment is determined; other deviation measures over time may be used instead of the standard deviation, for example variance and median absolute deviation.

The SoB score may be calculated in each of the three dimensions separately. These SoB scores are denoted herein with “mass_center_dev_x”, “mass_center_dev_y”, and “mass_center_dev_z” for the x, y, and z direction, respectively. Alternatively, the respective SoB scores might be combined into a single SoB score, or a single SoB score might be computed, which takes into account either only one certain direction or a plurality of directions.

The SoB score is related to the presence and severity of movement disorders of the test person that can, be related to neurodegenerative diseases such as one or more of Huntington's disease (HD), Parkinson's disease (PD), and Primary Lateral Sclerosis (PLS). The correlation is demonstrated using experimental data from a real-world setting described hereinafter.

For the SoB determination, optional data filtering, filtering in the sense of data smoothing, may be applied. For example, filtering can be applied to a series of tracked locations of each of the one or more anatomical landmarks, Filtering can be done by, for example, 2nd order of Savitzky-Golay smoothing, e.g. within a one second time window over the predefined time period during which depth image data and accordingly movement anatomical landmarks are recorded. Other forms of filtering include low pass filtering and computation of a moving average. For the clinical prove and data discussed and shown in the following, filtering was not applied.

### Clinical Prove and Data

The method 500 for determining a stability of body, SoB, score presented hereinabove was used to assess the whole-body balance of persons, being test subjects, from a real-world dataset. Four groups of subjects were measured: 50 patients with Huntington's Disease (HD), 30 patients with Parkinson's Disease (PD), 12 patients with Primary lateral sclerosis (PLS) and 14 healthy controls (CON), listed in the following table:

| GROUP | TOTAL | FEMALE |
|---|---|---|
| CON | 14 | 11 |
| HD | 50 | 20 |
| PD | 30 | 10 |
| PLS | 12 | 6 |

The subjects performed a 30-second balance test—standing still in front of a Kinect camera. The joint locations were recorded at a rate of 30 frames per second. Joint locations used were two ankles, two knees, two hips, two shoulders, two elbows, two wrists, spine middle, neck, and head. The raw data was used to calculate the SoB scores according to the formula provided hereinabove without any preprocessing. Wilcoxon rank-sum test was used for nonparametric pairwise comparison followed by a Benjamini-Hochberg correction for multiple comparisons. Effect size is calculated using Hedge's g due to the small sample size. A p-value of 0.05 was considered as significant for Wilcoxon rank-sum test. For effect size Cohen suggested that 0.2 be considered a small effect size, 0.5 represents a medium effect size and 0.8 a large effect size.

The SoB score in all three directions could distinguish the HD from CON, and HD from PLS with a significant p-value and large effect size. In z-direction which corresponds to the sagittal plane of the human body (120, 220, 320), the feature could separate all pairwise groups except for CON and PLS with significant p-values and medium or large effect size, which indicate the sensitivity of the proposed feature. It is generally observed that the CON group has significant smaller movement, or in other words, significant stability compared to the HD and PD groups. But the PLS groups has a similar stability in comparison with the CON group. For all the features of the HD group have significantly more movement or bigger deviation than the other three groups including even the PD group.

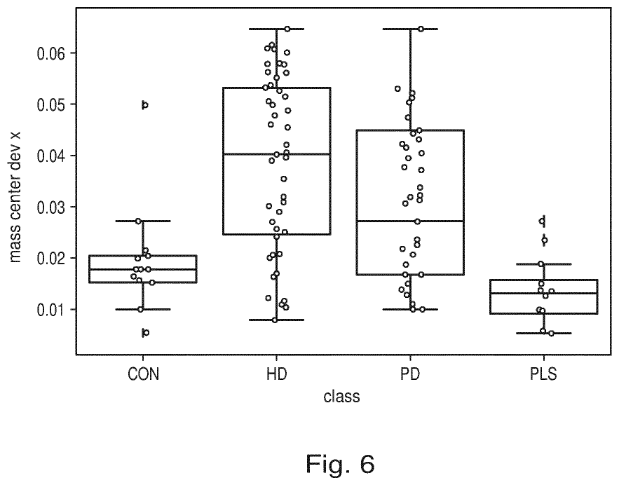

FIG. 6 shows the results in x-direction. After multiple comparison correction, the resulting adjusted p-values of Wilcoxon rank-sum test between groups are given in the following table:

| | CON | HD | PD | PLS |
|---|---|---|---|---|
| CON | −1.00 | | | |
| HD | 2.82 × 10⁻³ | −1.00 | | |
| PD | 3.50 × 10⁻² | 3.50 × 10⁻² | −1.00 | |
| PLS | 1.17 × 10⁻¹ | 1.01 × 10⁻⁴ | 3.81 × 10⁻³ | −1.00 |

The effect size of Hedge's g between groups is given in the following table:

| | CON | HD | PD | PLS |
|---|---|---|---|---|
| CON | 0.00 | | | |
| HD | −1.262 | 0.00 | | |
| PD | −0.762 | 0.492 | 0.00 | |
| PLS | 0.503 | 1.599 | 1.112 | 0.00 |

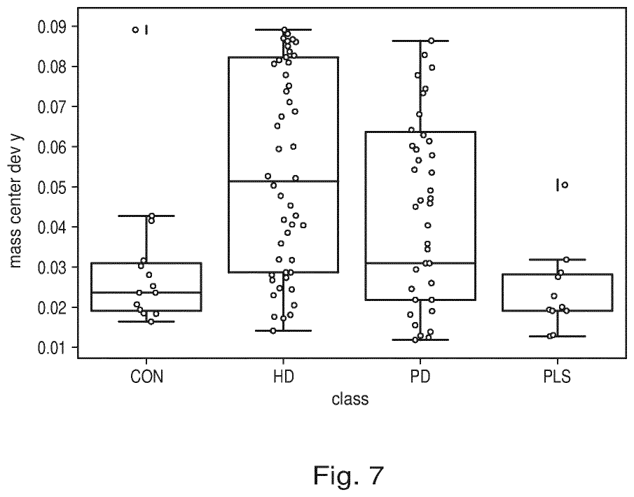

FIG. 7 shows the results in y-direction. The resulting p-value of Wilcoxon rank-sum test between groups are given in the following table:

| | CON | HD | PD | PLS |
|---|---|---|---|---|
| CON | −1.00 | | | |
| HD | 5.93 × 10⁻³ | −1.00 | | |
| PD | 3.02 × 10⁻¹ | 1.99 × 10⁻² | −1.00 | |
| PLS | 3.16 × 10⁻¹ | 1.40 × 10⁻³ | 4.03 × 10⁻³ | −1.00 |

The effect size of Hedge's g between groups is given in the following table:

| | CON | HD | PD | PLS |
|---|---|---|---|---|
| CON | 0.00 | | | |
| HD | −0.995 | 0.00 | | |
| PD | −0.444 | 0.563 | 0.00 | |
| PLS | 0.407 | 1.293 | 0.766 | 0.00 |

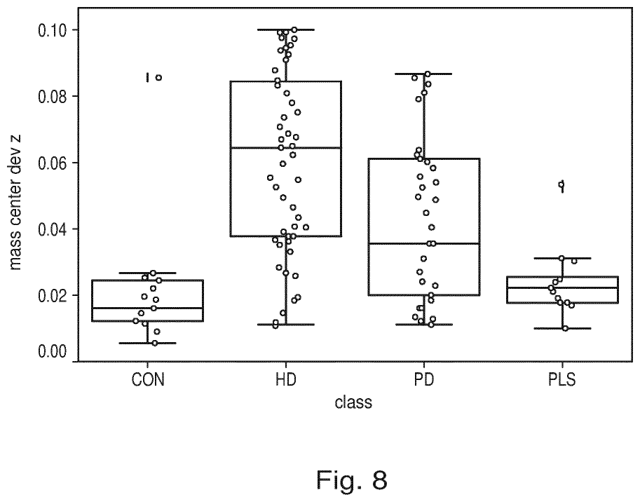

FIG. 8 shows the results in z-direction. The resulting p-value of Wilcoxon rank-sum test between groups are given in the following table:

| | CON | HD | PD | PLS |
|---|---|---|---|---|
| CON | −1.00 | | | |
| HD | 9.7 × 10⁻⁵ | −1.00 | | |
| PD | 5.87 × 10⁻³ | 5.29 × 10⁻³ | −1.00 | |
| PLS | 2.69 × 10⁻¹ | 1.51 × 10⁻⁴ | 4.89 × 10⁻² | −1.00 |

The effect size of Hedge's g between groups is given by:

| | CON | HD | PD | PLS |
|---|---|---|---|---|
| CON | 0.00 | | | |
| HD | −1.437 | 0.00 | | |
| PD | −0.802 | 0.734 | 0.00 | |
| PLS | −0.103 | 1.410 | 0.776 | 0.00 |

### Correlation of SoB Scores and Clinical Assessment for HD Group

The correlation between the Unified Huntington's Disease Rating Scale (UHDRS), more some specifically some of the characteristics included UHDRS, with SoB scores has been investigated for in total 8 HD patients. The UHDRS consist of four parts: Part 1 tests motor function including 31 items with a 5-point ordinal scale ranging from 0-4, part 2 tests the cognitive function, part 3 is a behavioral assessment, and part 4 assess the functional capacity, which is expressed by the Total Functional Capacity Score (TFC).

The SoB score has significant correlations (Spearman) with the total motor score from part 1 and TFC from part 4. Significant correlations were also found between SoB scores and two individual items related to the whole-body balance from part 1: maximal dystonia (trunk and extremities) and retropulsion pull test.

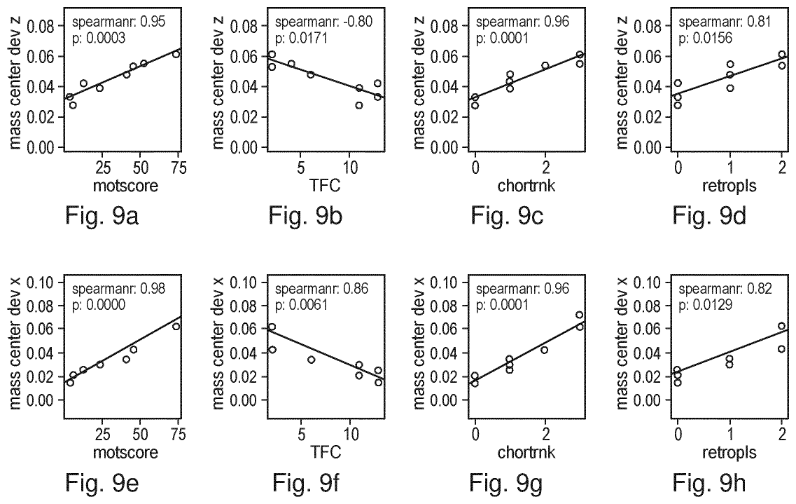

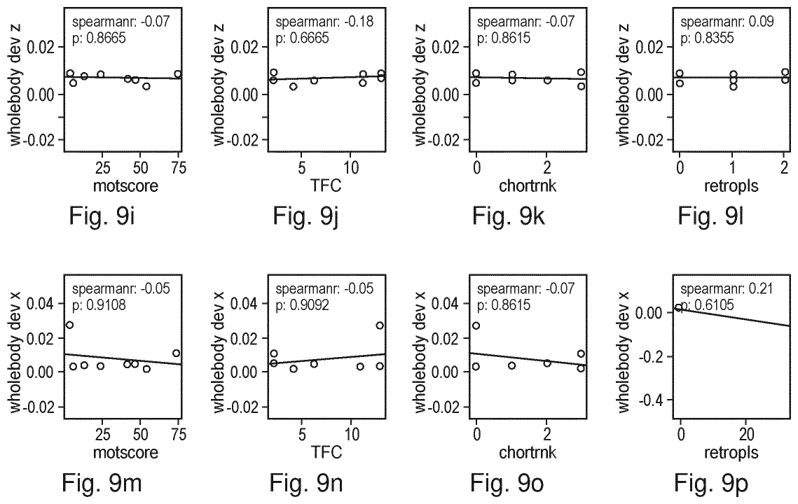

FIGS. 9a to 9h show the Spearman correlation between SoB scores in x- and z-direction (mass_center_dev_x, mass_center_dev_z) and the total motor score (motscore), the TFC, the maximal dystonia (chortrnk), and the retropulsion pull test (retropls) in scatterplots, with significance measures included in each plot. Only x- and z-directions are plotted as they represent the left and right instability, as well as the front and back instability while the y-direction is up and down which is less meaningful for a standing balance test.

Even though the sample size is small (only 8), such correlation is not observed by other balance assessments such as the deviation of the whole-body points (not relative to the spine base), shown in FIGS. 9i to 9p. These experimental data are shown to confirm and emphasize that the Spearman correlation observed for the SoB scores in FIGS. 9a to 9h is not due to the small sample size.

As the UHDRS test comprises 31 items, taking an UHDRS score requires a lot of time. In addition, the determined score is partially subjective and different examiners might assess identical performances differently. In contrast, the presented method is fast (predetermined test period is typically about 30 seconds), reproducible and objective. In addition, due to the short time span, the performance of a person might not significantly decrease as might be the case for longer tests.

Accordingly, the technology presented herein allows to assess the degree of a neurodegenerative disease, for instance by estimating TFC characteristics of UHDRS through the SoB score. For this purpose, Spearman correlation (Spearman's rank correlation), which is nonparametric and monotonic, is used as shown in the statistical data hereinabove. Furthermore, the disclosed technology allows to discriminate between movement disorders of different neurodegenerative or neuromuscular diseases.

While the present disclosure describes technologies for assessing whole-body balance with a focus on movement disorders that can, among others, be related to neurodegenerative diseases such as one or more of Huntington's disease (HD), Parkinson's disease (PD), and Primary Lateral Sclerosis (PLS), it should be noted that the disclosed technologies for assessing whole-body balance are not limited to determine the presence of movement disorders for the aforementioned diseases, but instead applies to all types of movement disorders.

Aspects of this disclosure can be implemented in digital circuits, computer-readable storage media, as one or more computer programs, or a combination of one or more of the foregoing. The computer-readable storage media can be non-transitory, e.g., as one or more instructions executable by a cloud computing platform and stored on a tangible storage device.

In this specification the phrase “configured to” is used in different contexts related to computer systems, hardware, or part of a computer program. When a system is said to be configured to perform one or more operations, this means that the system has appropriate software, firmware, and/or hardware installed on the system that, when in operation, causes the system to perform the one or more operations. When some hardware is said to be configured to perform one or more operations, this means that the hardware includes one or more circuits that, when in operation, receive input and generate output according to the input and corresponding to the one or more operations. When a computer program is said to be configured to perform one or more operations, this means that the computer program includes one or more program instructions, that when executed by one or more computers, causes the one or more computers to perform the one or more operations.

Unless otherwise stated, the foregoing alternative examples are not mutually exclusive, but may be implemented in various combinations to achieve unique advantages. In the foregoing description, the provision of the examples described, as well as clauses phrased as “such as,” “including” and the like, should not be interpreted as limiting embodiments to the specific examples; rather, the examples are intended to illustrate only one of many possible embodiments.

1. A computer-implemented method for analyzing whole-body balance of a person, the method comprising:
obtaining depth image data of a person's body during a predefined time period;
tracking locations of a plurality of anatomical landmarks of the body based on the depth image data;
estimating a mass center of the body based on a tracked location of each of one or more of the anatomical landmarks; and
determining a stability of body, SoB, score based on a deviation of the estimated mass center over the predefined time period, wherein the SoB score is indicative of the whole-body balance.

2. The method of claim 1, wherein the center of mass is estimated by a sum vector of each of the one or more anatomical landmarks relative to a predefined anatomical landmark in one, two, or three spatial dimensions.

3. The method of claim 1, wherein each anatomical landmark corresponds to a joint of the person's skeleton and the predefined anatomical landmark corresponds to a spine base, a hip center, or a pelvis center of the body.

4. The method of claim 1, wherein the SoB score is a standard deviation of the center of mass estimation over the predefined time period.

5. The method of claim 1, further comprising:
excluding one or more of the plurality of anatomical landmarks based on a noise level of the tracked locations of respective anatomical landmarks; and/or
smoothing a series of tracked locations of one or more of the plurality of anatomical landmarks.

6. The method of claim 1, further comprising recording a plurality of SoB scores of the person over time in a data store to assess, using Spearman correlation, a progression of neurodegenerative or neuromuscular disease of the person and/or to monitor the effect of one or more treatment schemes such as medication and physiotherapy.

7. The method of claim 1, wherein obtaining depth image data of the person's body includes capturing the depth image data using a motion sensor.

8. A system for analyzing whole-body balance of a person, the system comprising:
a motion sensor for capturing depth image data of a person's body;
a processor in communication with the motion sensor, wherein the processor is configured to perform operations comprising:
tracking locations of a plurality of anatomical landmarks of the body based on the depth image data;
estimating a mass center of the body based on a tracked location of each of one or more of the anatomical landmarks; and
determining a stability of body, SoB, score based on a deviation of the estimated mass center along the predefined time period, wherein SoB score is indicative of the whole-body balance.

9. The system of claim 8, further comprising a data store communicatively connected to the processor;
wherein the processor is further configured to perform the step of recording a plurality of SoB scores of a person over time in the data store to assess, using Spearman correlation, a progression of neurodegenerative disease and/or to monitor the effect of one or more treatment schemes such as medication and physiotherapy.

10. The system according to claim 8, wherein the motion sensor is a 3D motion sensor including an infrared camera, a Kinect sensor, or a RealSense sensor.

11. The system of claim 8, wherein the motion sensor is included in a mobile computing device.

12. The system of claim 11, wherein the processor is included in a remote computer and wherein the depth image data is communicated by the mobile computing device via a network to the remote computer.

13. A server computing device comprising:
a network adapter; and
a processor adapted to perform the steps:
receiving depth image data of a person's body via the network adapter;
tracking locations of a plurality of anatomical landmarks of the body based on the depth image data;
estimating a mass center of the body based on a location of each of one or more of the anatomical landmarks; and
determining a stability of body, SoB, score based on a deviation of the estimated mass center along the predefined time period, wherein the SoB score is indicative of the whole-body balance of the person's body.

14. (canceled)

15. (canceled)

16. The system of claim 8, wherein the center of mass is estimated by a sum vector of each of the one or more anatomical landmarks relative to a predefined anatomical landmark in one, two, or three spatial dimensions.

17. The system of claim 8, wherein each anatomical landmark corresponds to a joint of the person's skeleton and the predefined anatomical landmark corresponds to a spine base, a hip center, or a pelvis center of the body.

18. The system of claim 8, wherein the SoB score is a standard deviation of the center of mass estimation over the predefined time period.

19. The system of claim 8, wherein the operations further comprise:
excluding one or more of the plurality of anatomical landmarks based on a noise level of the tracked locations of respective anatomical landmarks; and/or
smoothing a series of tracked locations of one or more of the plurality of anatomical landmarks.
